(19) United States
(12) Patent Application Publication
(10) Pub. No.: US 2010/0015610 A1
(43) Pub. Date: Jan. 21, 2010

(54) DIAGNOSTIC SCREENING METHODS FOR DISORDERS OF THE ENDOPLASMIC RETICULUM-TO-GOLGI TRAFFICKING OF PROTEINS

(75) Inventor: Simeon Boyd, Davis, CA (US)

Correspondence Address: EDWARDS ANGELL PALMER & DODGE LLP, P.O. BOX 55874, BOSTON, MA, 02205, US

(73) Assignee: The Johns Hopkins University, Baltimore, MD (US)

(21) Appl. No.: 12/227,733

(22) PCT Filed: May 22, 2007
(86) PCT No.: PCT/US2007/012154

§ 371 Date: Apr. 8, 2009

Related U.S. Application Data
(60) Provisional application No. 60/802,574, filed on May 22, 2006.

(51) Int. Cl. C12Q 1/68; G01N 33/574

(52) U.S. Cl. 435/6; 435/7.4

(57) ABSTRACT

The invention relates to methods of diagnosing Cranio-lenticulo-sutural dysplasia and other disorders that occur as a result of defective endoplasmic reticulum-to-Golgi trafficking using immunofluorescence based screening tests using antibodies against protein disulfide isomerase.

### PRIORITY CLAIM

This application claims priority from U.S. Provisional Application No. 60/802,574, filed May 22, 2006, which is incorporated herein by reference.

### GOVERNMENT SUPPORT

Funding for this invention was provided in part by the Government of the United States of America National Institutes of Health—NIDCR Grants DE 16342 and DE00462. The Government has certain rights in this invention.

### FIELD OF THE INVENTION

The invention relates to methods of diagnosing Cranio-lenticulo-sutural dysplasia and other disorders that occur as a result of defective Endoplasmic Reticulum-to-Golgi trafficking of proteins using immunofluorescence based diagnostic tests and antibodies directed against protein disulfide isomerase.

### BACKGROUND OF THE INVENTION

Approximately one-third of all cellular proteins are synthesized in association with ER membranes. These proteins are retained in the ER lumen for proper folding, oligomerization and post-translational modifications. COPII-coated transport vesicles represent the first step of the intracellular secretory pathway responsible for the traffic of properly modified proteins from the ER to the ER-Golgi intermediate compartment (ERGIC) and ultimately to the cis-Golgi complex (reviewed in^{2,3}). The secretory pathway in eukaryotic cells has been extensively studied and well characterized, mostly by genetic and biochemical analyses of temperature-sensitive conditional mutants of Saccharomyces cerevisiae⁴⁻⁶. The COPII coat is a polymer complex formed by at least five well characterized proteins—SARI, SEC23, SEC24, SEC 13, and SEC31. Multiple isoforms of these proteins have been identified, and it has been shown that a distinct coat structure is responsible for the ER export of specific sets of secretory proteins⁷⁻¹⁰. The formation of the COPII complex is initiated when the small GTPase SARI is anchored to the cytosolic surface of the ER and activated by GDP/GTP exchange by SEC 12, an integral membrane glycoprotein^{11,12} Activated SARI directly binds the SEC23/SEC24 complex, a heterodimer protein responsible for membrane cargo protein recognition. Cargo molecules tethered to SARI, SEC23/SEC24 are coated by the SEC 13/SEC311 complex forming buds and vesicles destined for the Golgi apparatus. In addition to its role in coating and stabilizing COPE vesicles and selective recruitment of cargo proteins in the budding vesicle¹³, SEC23 acts as a SARI-specific GTPase activating protein (GAP) that hydrolyzes the SARI-bound GTP which triggers uncoating of the vesicles and exposure of SNARE proteins needed for vesicle fusion to an acceptor compartment. The SEC24 component of the COPA coat determines the specificity of the cargo proteins that are to be exported from the ER^{9,14}. The precise sites of interaction of SARI, SEC23, and SEC24 have been determined, but it remains unclear where exactly SEC 13 and SEC31 bind^{15,16}. Conditional yeast Sec23 mutants demonstrate distention of the terminal portions of the ER at restrictive temperatures due to a block of the secretory pathway at the ER exit sites⁴. Similar cellular changes are present in C. elegans SEC-23 mutants that exhibit mislocalization of the collagen DPY-7 and defects of morphogenesis and exoskeleton formation¹⁷.

Cranio-lenticulo-sutural dysplasia (CLSD, OMIM 607812) was originally observed and described in an inbred Saudi Arabian family in which five males and one female had wide-open and late-closing anterior fontanels, characteristic facial appearance with prominent hypertelorism (FIG. 1a), early onset Y-shaped sutural cataracts, and mild skeletal and connective tissue defects. We previously showed linkage to chromosome 14g13-q21 by genome-wide scan with the maximum lod score of 4.58 at GATA126A04¹.

Genes within the candidate region were screened by direct sequencing of cDNA generated by RT-PCR We observed a c.1144T>C transition in SEC23A that segregated in a homozygous form in all affected individuals and was not present in 600 control chromosomes (FIG. 1b). Three-dimensional homology modeling of SEC23A based on the previously determined crystal structure of yeast Sec23¹⁶ placed the mutant F382L residue in the trunk domain of the protein and predicted an abnormal protein folding. The F382L substitution involved a residue invariably conserved in at least ten species (FIG. 1c).

Traditional methods for the diagnosis of heritable diseases have depended on either the identification of abnormal gene products (e.g., sickle cell anemia) or an abnormal phenotype (e.g., mental retardation). These methods are of limited utility for heritable diseases with late onset and no easily identifiable phenotypes, such as, for example, Alzheimer's disease. With the development of genetic testing, it is now possible to identify gene mutations which indicate a propensity to develop disease, even when the disease is of polygenic origin. The number of diseases that can be diagnosed by molecular biological methods continues to grow with increased understanding of the genetic basis of multifactorial disorders (see e.g., U.S. Pat. Nos. 4,582,788; 5,110,920; 4,801,531; 4,666,828; and 5,268,267).

The ability to diagnose individuals affected by ER-to-Golgi trafficking of proteins using a rapid and cost effective diagnostic method and kit would allow physicians to focus on treatment measures much earlier and with greater certainty for which disease or disorder they are treating. As such, it would be highly desirable to develop diagnostic assays which would detect individuals with diseases and/or disorders associated with defects in ER-to-Golgi trafficking of proteins.

### SUMMARY OF THE INVENTION

The present invention provides inter alia for novel diagnostic methods of Cranio-lenticulo-sutural dysplasia and other disorders that occur as a result of defective endoplasic reticulum(ER)-to-Golgi trafficking using immunofluorescence based screening tests using antibodies against protein disulfide isomerase. It also provides kits for the detection of such disorders.

Cranio-lenticulo-sutural dysplasia (CLSD) is a novel autosomal recessive syndrome with late-closing fontanels, sutural cataracts, facial dysmorphisms, and skeletal defects mapped to chromosome 14g13-g21¹. Using a positional cloning approach we identified F382L missense mutation in SEC23A segregating with this syndrome. SEC23A is an essential component of the COP-coated vesicles that transport secretory proteins from the endoplasmic reticulum (ER) to the Golgi complex. Electron microscopy and immunofluorescence (IF) documented gross dilatation of the ER inpatient fibroblasts. The cells also exhibited cytoplasmic mislocalization of SEC31 and delayed secretion of COL1A1. Cell-free vesicle budding assays demonstrated that the F382L mutation results in loss of SEC23A function. A phenotype reminiscent of CLSD was observed in Zebrafish embryos injected with sec23a blocking morpholinos. We have discovered that a secretory defect of a distinct set of cargo proteins required for normal morphogenesis accounts for CLSD.

Specifically, the present invention relates to methods of diagnosing a patient with a disease or disorder that is associated with defective Endoplasmic Reticulum-to-Golgi trafficking of proteins comprising:

a) culturing primary fibroblast cells from a patient;

b) plating the cultured fibroblast cells on a transparent structural support and treating the cells with a permeabilizing agent;

c) incubating the cells with a first antibody of animal species A which is directed to protein disulfide isomerase;

d) incubating the cells with a second antibody of animal species B which is conjugated with a fluorescent label and directed to the first antibody of animal species A; and

e) visualizing the cells under a fluorescence microscope and identifying distended Endoplasmic Reticulum with abundant vacuolar structures wherein the presence of distended Endoplasmic Reticulum is diagnostic of a disease or disorder associated with defective Endoplasmic Reticulum-to-Golgi trafficking of proteins for the patient.

The invention further relates to added analysis wherein following the method described above, the RNA of the fibroblast cells is extracted from the cells and quantitative RT-PCR analysis is conducted for a specific isoform of the X-Box-Binding Protein 1 (XBP1) gene to determine Unfolded Protein response (UPR) activation, which if UPR is not activated, an Endoplasmic Reticulum-to Golgi disease or disorder is confirmed.

The methods of the present invention are particularly appropriate for diagnosing Cranio-lenticulo-sutural dysplasia

In certain embodiments of the invention the first antibody of animal species A is a rabbit antibody and the second antibody of animal species B is a goat antibody. Each type of antibody may be polyclonal or monoclonal. In addition, the fluorescent label may be fluorescein isothiocyanate or Texas red goat antibody.

In still other embodiments of the invention first antibody of animal species A which is directed to protein disulfide isomerase is commercially available or it can be developed by traditional methods of polyclonal or monoclonal antibody development.

Still further, another embodiment of the present invention relates to methods of diagnosing a patient with a disease or disorder that is associated with defective Endoplasmic Reticulum-to-Golgi tracking of proteins comprising:

a. culturing primary fibroblast cells from a patient;

b. plating the cultured fibroblast cells on a transparent structural support and treating the cells with a permeabilizing agent;

c. incubating the cells with a fluorescent labeled antibody which is directed to protein disulfide isomerase; and

d. visualizing the cells under a fluorescence microscope and identifying distended Endoplasmic Reticulum with abundant vacuolar structures wherein the presence of distended Endoplasmic Reticulum is diagnostic of a disease or disorder associated with defective Endoplasmic Reticulum-to-Golgi trafficking of proteins for the patient. This method can be augmented wherein the RNA of the fibroblast cells is extracted from the cells and quantitative RT-PCR analysis is conducted for a specific isoform of the X-Box-Binding Protein 1 (XBP 1) gene to determine Unfolded Protein response (UPR) activation, which if UPR is not activated, a Endoplasmic Reticulum-to Golgi disease or disorder is confirmed.

Another embodiment of the present invention relates to a kit for diagnosing a patient with a disease or disorder that is associated with defective Endoplasmic Reticulum-to-Golgi trafficking of proteins comprising a first antibody of animal species A which is directed to protein disulfide isomerase, a second antibody of animal species B which is conjugated with a fluorescent label and directed to the first antibody of animal species A and instructions for use in performing the diagnostic test.

Other embodiments and advantages of the invention are set forth in part in the description which follows, and will be obvious from this description, or may be learned from the practice of the invention.

### BRIEF DESCRIPTION OF THE FIGURES

FIG. 1. (a) Craniofacial features of CLSD (clockwise from top left)—age progression of an affected male at age of 4 and 9 with cranial radiogram demonstrated persistent ossification defect; cranial radiogram of an affected female shows wide-open anterior fontanel at age 28 months, her facial appearance at age 7 and lateral view with marked forehead hyperpigmentation at 28 months; (b) c.1144T>C transition in exon 10 of SEC23A was present in a homozygous form in all six affected individuals and not found in 600 control chromosomes; (c) Protein sequence homology analysis demonstrates that F382 is invariably conserved in at least ten species, including A. thaliana.

FIG. 2. The PDI Immunofluorescence demonstrates fine reticular appearance of ER in the wild-type fibroblasts (a) as opposed to the marked dilatation of the ER in the mutant fibroblasts (b and c); (c) is a higher magnification of the area outlined in (b) showing clustered vacuolar structures; Immunofluorescence of homozygous mutant cells visualizing pro-collagen COL1A1 (d) shows similar vacuolar structures; Immunofluorescence with SEC31 antibody produced punctate staining mostly in the perinuclear region in control (e) and diffuse cytoplasmic mislocalization in mutant fibroblasts (f).

FIG. 3. Electron microscopy of control (a), SEC23A heterozygous (b) and SEC23A homozygous mutant (c) fibroblasts shows the appearance of a normal, moderately dilated, and grossly dilated endoplasmic reticulum, respectively.

FIG. 4. Immunoblot analysis of COL1A1 secretion (a) comparing extracellular and intracellular fractions confirms that heterozygous SEC23A±fibroblasts secrete more COL1A1 than the homozygous SEC23A−/− fibroblasts. (b) Quantification of collagen secretion in the presence and absence of cyclohexamide (Cly) for 6.5 h.

FIG. 5. In vitro studies of F382L SEC23A. A liposome binding assay, (a), demonstrates that the mutant protein is able to bind synthetic membranes when SARIB is activated (triphosphate-bound), similarly to the wild-type protein; A vesicle formation assay, (b), demonstrates that F382L SEC23A has markedly lower activity for generation of cargo-containing vesicles compared to the wild-type protein; Ribophorin I is an ER-resident protein serving as a negative control, whereas Sec22b and p58 are COPA cargo proteins. ATPr, ATP regeneration system.

FIG. 6. Developmental expression of sec23a and loss of function phenotype in zebrafish; RT-PCR analysis detected sec23a transcript as early as in the 1-cell stage (a), suggesting its presence as a maternal transcript; Whole mount in situ hybridization analysis shows that transcripts are present at very low levels from the 1-cell stage (b) through the 1000-cell mid-blastula transition stage (c), and up to the bud stage (not shown); At the 12-somite stage (d), weak but distinct expression is detected in the developing notochord; Notochord expression is strongest in the 1 dpf embryo (e) and is not longer detectible in 2 dpf embryos notochord; Weak sec23a expression is evident at 2 dpf and is clearly observed in the neurocranial and viscerocranial cartilages of the head in 3 dpf embryos (f, ventral view and g, lateral view), the cranial project and bulge of the otic vesicle (h), as well as the scapulocoracoid, postcoracoid process and distal edge of the endoskeletal disc of the pectoral fin (i); Loss-of-function 5 dpf morphants exhibit reduced body length and dorsal curvature (j) compared to wildtype larvae (k), kinked pectoral fins (l, m) due to a larger non-cartilaginous fin segment at the distal edge (n, o), and malformation/dysgenesis of the head cartilages (p, q). MO, morphant; WT, wildtype; act, actinotrichs; aud, auditory capsule; cbl to 5, ceratobranchial arches 1 to 5; ch, ceratohyal arch; cnb, cranial bulge; cnp, cranial project; ed, endoskeletal disc; ep, ethmoid plate; m, Meckel's cartilage; nc, notochord; pf, pectoral fin; pop, postcoracoid process; sco, scapulocoracoid.

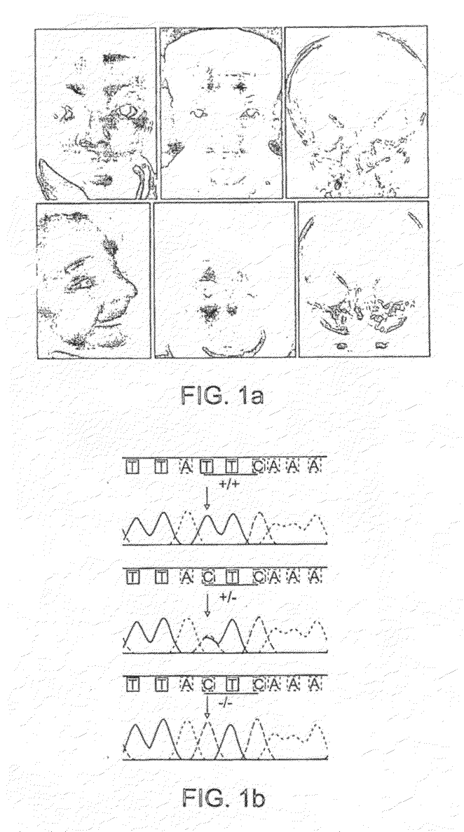

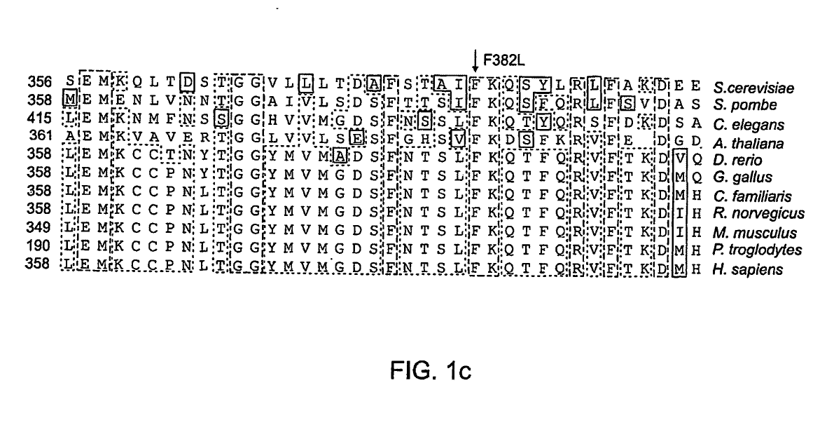

### DETAILED DESCRIPTION OF THE INVENTION

As embodied and broadly described herein, the present invention is directed to methods for the novel diagnostic methods of Cranio-lenticulo-sutural dysplasia and other disorders that occur as a result of defective endoplasic reticulum-to-Golgi trafficking using immunofluorescence based screening tests using antibodies against protein disulfide isomerase. It also provides kits for the detection of such diseases/disorders.

CLSD has been delineated as a novel dysmorphic genetic syndrome with characteristics of a skeletal dysplasia and were able to identify its genetic cause. The experiments described in this application demonstrate that CLSD occurs as a result of defective COPII mediated ER export due to abnormal SEC23A. As a result, COL 1A1 and, likely, other secretory proteins accumulate and distend the ER and ultimately lead to the clinical manifestations of CLSD. The relatively mild phenotype of the affected individuals suggests functional redundancy within the COPII trafficking pathway. Further studies of the mutant cells and/or a SEC23A animal model will allow more precise identification of the cargo proteins retained in the ER as a result of mutations in SEC23A. The characteristic phenotype of the SEC23A mutant cells suggests that screening methods may be developed that would allow the identification of other human disorders caused by defects of ER-to-Golgi trafficking.

Analysis of the orthologous sec23a gene in the zebrafish revealed an anatomical and morphological correlation with the human CLSD phenotype, providing further evidence that loss of SEC23A function is responsible for this genetic syndrome.

Although the ER-to-Golgi trafficking has been extremely well characterized by both genetic and biochemical methods very few human disorders²²⁻²⁴ have been attributed to defects of its individual components. The functional redundancy of the COPII pathway is likely to lead to non-lethal phenotypes that have escaped classification. A systematic survey of tissues from similar bone morphogenesis diseases may uncover other novel mutant alleles of the COPE machinery.

### Protein Disulfide Isomerase^{29,30,31}

Protein disulfide isomerase (E.C. 5.3.4.1) is an abundant, soluble protein found in the lumen of the endoplasmic reticulum (ER) of eukaryotic cells. It is directed across the ER membrane at biosynthesis by a classical signal sequence and retained in the ER by a KDEL sequence on its carboxy terminus. The major role for protein disulfide isomerase is thought to be the formation and rearrangement of disulfide bonds. Protein disulfide isomerase is found in cells mainly as a homodimer.

Formation of a disulfide bond via the action of protein disulfide isomerase involves both oxidation and isomerization steps. The most plausible models are shown below (to be added at some point). The active site of protein disulfide isomerase resembles that of thioredoxin. Active site cysteine groups of PDI form mixed disulfides with other proteins.

Protein disulfide isomerase is localized in the lumen of the ER at a concentration of approximately 10 mg/ml and is enriched in secretory cells.

Protein disulfide isomerase is a component of prolyl-4-hydroxylase (an alpha2beta2 tetramer where the beta subunits are protein disulfide isomerase), an enzyme that catalyzes the hydroxylation of prolines in procollagen during the synthesis of collagen. Protein disulfide isomerase is also a component of microsomal triacylglycerol transfer protein (a heterodimer of PDI and a 97 kDa subunit), that is believed to catalyze the transfer of neutral lipid onto nascent lipoprotein particles. When complexed with either prolyl-4-hydroxylase or the microsomal triacylglycerol transfer protein, protein disulfide isomerase does not show isomerase activity suggesting that it may be acting to stabilize the complexes.

### Antibodies Against Protein Disulfide Isomerase

In an embodiment, the present invention provides for antibodies against Protein Disulfide Isomerase (PDI) which may accumulate in the Endoplasmic Reticulum. Such antibodies are useful in the diagnostic methods of the present invention. These antibodies can be obtained commercially, such as from Biocompare, Inc., South San Francisco, Calif. or alternative they can be developed using standard techniques for developing polyclonal and monoclonal antibodies described below.

Antibodies of the invention include polyclonal antibodies, monoclonal antibodies, and fragments of polyclonal and monoclonal antibodies.

PDI can also be used to produce antibodies which are immunoreactive or bind to epitopes of the PDI polypeptides. Antibody which consists essentially of pooled monoclonal antibodies with different epitopic specificities, as well as distinct monoclonal antibody preparations are provided. Monoclonal antibodies are made from antigen containing fragments of the protein by methods well known in the art (Kohler, et al., Nature, 256:495, 1975; Current Protocols in Molecular Biology, Ausubel, et al., ed., 1989).

The term “antibody” as used in this invention includes intact molecules as well as fragments thereof, such as Fab, F(ab′)₂, and Fv which are capable of binding the epitopic determinant. These antibody fragments retain some ability to selectively bind with its antigen and are defined as follows:

(1) Fab, the fragment which contains a monovalent antigen-binding fragment of an antibody molecule can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;

(4) Fv, defined as a genetically engineered fragment containing the variable region of the light chain and the variable region of the heavy chain expressed as two chains; and

(5) Single chain antibody (“SCA”), defined as a genetically engineered molecule containing the variable region of the light chain, the variable region of the heavy chain, linked by a suitable polypeptide linker as a genetically fused single chain molecule.

Methods of making these fragments are known in the art. (See for example, Harlow and Lane, Antibodies: A Laboratory Manual, Cold Spring Harbor Laboratory, New York (1988), incorporated herein by reference).

As used in this invention, the term “epitope” means any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics.

Antibodies which bind to the PDI polypeptide can be prepared using an intact polypeptide or fragments containing small peptides of interest as the immunizing antigen. The polypeptide or a peptide used to immunize an animal can be derived from translated cDNA or chemical synthesis which can be conjugated to a carrier protein, if desired. Such commonly used carriers which are chemically coupled to the peptide include keyhole limpet hemocyanin (KLH), thyroglobulin, bovine serum albumin (BSA), and tetanus toxoid. The coupled peptide is then used to immunize the animal (e.g., a mouse, a rat, or a rabbit).

If desired, polyclonal or monoclonal antibodies can be further purified, for example, by binding to and elution from a matrix to which the polypeptide or a peptide to which the antibodies were raised is bound. Those of skid in the art will know of various techniques common in the immunology arts for purification and/or concentration of polyclonal antibodies, as well as monoclonal antibodies (See for example, Coligan, et al., Unit 9, Current Protocols in Immunology, Wiley Interscience, 1994, incorporated by reference).

It is also possible to use the anti-idiotype technology to produce monoclonal antibodies which mimic an epitope. For example, an anti-idiotypic monoclonal antibody made to a first monoclonal antibody will have a binding domain in the hypervariable region which is the “image” of the epitope bound by the first monoclonal antibody.

The preparation of polyclonal antibodies is well-known to those skilled in the art. See, for example, Green et al., Production of polyclonal Antisera, in IMMUNOCHEMICAL PROTOCOLS (Manson, ed.), pages 1-5 (Humana Press 1992); Coligan et al., Production of Polyclonal Antisera in Rabbits, Rats, Mice and Hamsters, in CURRENT PROTOCOLS IN IMMUNOLOGY, section 2.4.1 (1992), which are hereby incorporated by reference.

The preparation of monoclonal antibodies likewise is conventional. See, for example, Kohler & Milstein, Nature 256:495 (1975); Coligan et al., sections 2.5.1-2.6.7; and Harlow et al., ANTIBODIES: A LABORATORY MANUAL, page 726 (Cold Spring Harbor Pub. 1988), which are hereby incorporated by reference. Briefly, monoclonal antibodies can be obtained by injecting mice with a composition comprising an antigen, verifying the presence of antibody production by removing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan et al., sections 2.7.1-2.7.12 and sections 2.9.1-2.9.3; Barnes et al., Purification of Immunoglobulin G (IgG), in METHODS IN MOLECULAR BIOLOGY, VOL. 10, pages 79-104 (Humana Press 1992). Methods of in vitro and in vivo multiplication of monoclonal antibodies is well-known to those skilled in the art. Multiplication in vitro may be carried out in suitable culture media such as Dulbecco's Modified Eagle Medium or RPMI 1640 medium, optionally replenished by a mammalian serum such as fetal calf serum or trace elements and growth-sustaining supplements such as normal mouse peritoneal exudate cells, spleen cells, bone marrow macrophages. Production in vitro provides relatively pure antibody preparations and allows scale-up to yield large amounts of the desired antibodies. Large scale hybridoma cultivation can be carried out by homogenous suspension culture in an airlift reactor, in a continuous stirrer reactor, or in immobilized or entrapped cell culture. Multiplication in vivo may be carried out by injecting cell clones into mammals histocompatible with the parent cells, e.g., syngeneic mice, to cause growth of antibody-producing tumors. Optionally, the animals are primed with a hydrocarbon, especially oils such as pristane (tetramethylpentadecane) prior to injection. After one to three weeks, the desired monoclonal antibody is recovered from the body fluid of the animal.

Alternatively, a diagnostically useful anti-PDI antibody may be derived from a “humanized” monoclonal antibody. Humanized monoclonal antibodies are produced by transferring mouse complementary determining regions from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Nat'l Acad. Sci. USA 86:3833 (1989), which is hereby incorporated in its entirety by reference. Techniques for producing humanized monoclonal antibodies are described, for example, by Jones et al., Nature 321: 522 (1986); Riechmann et al., Nature 332: 323 (1988); Verhoeyen et al., Science 239: 1534 (1988); Carter et al., Proc. Nat'l Acad. Sci. USA 89: 4285 (1992); Sandhu, Crit. Rev. Biotech. 12: 437 (1992); and Singer et al., J. Immunol. 150: 2844 (1993), which are hereby incorporated by reference

Antibodies of the invention also may be derived from human antibody fragments isolated from a combinatorial immunoglobulin library. See, for example, Barbas et al., METHODS: A COMPANION TO METHODS IN ENZYMOLOGY, VOL. 2, page 119 (1991); Winter et al., Ann. Rev. Immunol. 12: 433 (1994), which are hereby incorporated by reference. Cloning and expression vectors that are useful for producing a human immunoglobulin phage library can be obtained, for example, from STRATAGENE Cloning Systems (La Jolla, Calif.).

In addition, antibodies of the present invention may be derived from a human monoclonal antibody. Such antibodies are obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13 (1994); Lonberg et al., Nature 368:856 (1994); and Taylor et al., Int. Immunol. 6:579 (1994), which are hereby incorporated by reference.

Antibody fragments of the present invention can be prepared by proteolytic hydrolysis of the antibody or by expression in E. Coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using papain produces two monovalent Fab fragments and an Fc fragment directly. These methods are described, for example, by Goldenberg, U.S. Pat. Nos. 4,036,945 and 4,331,647, and references contained therein. These patents are hereby incorporated in their entireties by reference: See also Nisonhoff et al., Arch. Biochem. Biophys. 89:230 (1960); Porter, Biochem. J. 73:119 (1959); Edelman et al., METHODS IN ENZYMOLOGY, VOL. 1, page 422 (Academic Press 1967); and Coligan et al. at sections 2.8.1-2.8.10 and 2.10.1-2.10.4.

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

For example, Fv fragments comprise an association of V_{H} and V_{L} chains. This association may be noncovalent, as described in Inbar et al., Proc. Nat'l Acad. Sci. USA 69:2659 (1972). Alternatively, the variable chains can be linked by an intermolecular disulfide bond or cross-linked by chemicals such as glutaraldehyde. See, e.g., Sandhu, supra. Preferably, the Fv fragments comprise V_{H} and V V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by Whitlow et al., METHODS: A COMPANION TO METHODS IN ENZYMOLOGY, VOL. 2, page 97 (1991); Bird et al., Science 242:423-426 (1988); Ladner et al., U.S. Pat. No. 4,946,778; Pack et al., Bio/Technology 11: 1271-77 (1993); and Sandhu, supra.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick et al., METHODS: A COMPANION TO METHODS IN ENZYMOLOGY, VOL. 2, page 106 (1991).

### Fibroblast Culturing

The skin biopsy is accomplished, as is well known in the art, by harvesting from a patient a small patch of skin and fat, and then isolating the endothelial cells from the capillaries and the fibroblasts from the dermis. Blood vessel biopsies can be taken by removing a small segment of a peripheral vein or artery, preferably a jugular vein (or similar superficial vessel). Alternatively, a small segment can be harvested endoscopically (via a catheter) or by dissecting out a deep vessel. Mesothelial cells harvested from fat can be used in place of endothelial cells.

Once a suitable biopsy is taken, fibroblasts must be isolated and expanded to obtain purified cultures. However, reasonably low levels of other cell types may exist in the purified cultures. Fibroblasts can be isolated from the biopsy by several different well-known techniques. The easiest is manual dissection of skin or blood vessels to separate the fibroblast-containing tissue. For skin biopsies, the dermis must be isolated, taking care to remove hair follicles which are a source of keratinocyte contamination. For blood vessel biopsies, the adventitia must be isolated from the media and endothelial layers. Fibroblasts can be harvested from this portion of the tissue explant by cell outgrowth or by enzymatically digesting the explant and plating the digested tissue.

Fibroblasts can also be isolated by varying the culture conditions to favor their growth. Surface material choice (glass versus plastic) or surface preparation (gelatin or fibronectin coating) can be selected to favor fibroblast proliferation. Likewise, media additives and pH can be adjusted to promote a preference for endothelial cell or fibroblast proliferation. After a few passages, the cell population will be sufficiently pure. Fibroblasts may also be isolated by flow cytometry. In practice, fibroblasts are more difficult to sort by this technique, as there are no clear antibodies that are unique to fibroblasts.

After seeding, the first maturation and proliferation phase of the sheet growth begins. Fibroblasts will proliferate in almost any serum-containing cell culture media. A preferred media to optimize the fibroblast proliferation rate and the production of extracellular matrix proteins (which provide mechanical strength to the sheet) is DMEM and Hams F12 in a 1:1 ratio, supplemented with 10% fetal calf serum and antibiotics. A preferred embodiment also includes ascorbic acid or other ascorbate derivatives because they accelerate the production of the extracellular matrix proteins.

There are several growth conditions that must be maintained during sheet growth in module 1. First, the media pH should be maintained in a range between about 5 and 9, preferably approximately 7.4. Second, the media temperature should be maintained in a range between about 25° C. and 45° C., preferably approximately 37° C. Third, a sterile air environment should be employed, preferably including up to 20% CO₂ In addition, an adequate media exchange rate must be maintained to prevent exhausting critical media constituents.

Before being cultured with antibodies, the cells can be treated with a permeabilizing agent, such as 0.5% Triton-X 100, for a short period of time (i.e., 1 to 10 minutes) room temperature which will allow for the penetration of the antibodies into the cell and the cell's ER.

### Fluorescent Labels

The criteria for selecting an appropriate fluorescent label are that it must not hinder binding by the antibody to the PDI, i.e, the specificity and selectivity of the antibody, and that it must be detectable in the visible spectrum.

Examples of suitable fluorescent labels include fluorescein, 5,6-carboxymethyl fluorescein, Texas red, nitrobenz-2-oxa-1,3-diazol-4-yl (NBD), coumarin, dansyl chloride, and rhodamine. The preferred label is fluorescein (5-carboxyfluorescein-N-hydroxysuccinimide ester) or rhodamine (5,6-tetramethyl rhodamine). These can be obtained from a variety of commercial sources, including Molecular Probes, Eugene, Oreg. and Research Organics, Cleveland, Ohio.

It is important that the fluorescent label be attached to the antibody at an appropriate site so as to not interfere with binding activity of the antibody nor with fluorescence of the label. It is also important to choose a label which emits detectable fluorescence at the desired wavelength(s), under the conditions under which it is to be detected, including pH, ionic strength, polarity of the solution (for example, water versus alcohol versus ethyl acetate), and amount and type of tissue present (for example, brain tissue versus kidney tissue versus lung tissue versus heart).

Advantages to the use of fluorescence include the rapidity with which results can be obtained ( milliseconds), allowing use of the label in following binding over time, in contrast to radioactivity, which requires reaching equilibrium (e.g., 60 minutes), then stopping of the reaction (15 to 60 seconds), then a relatively lengthy process (hours to days to months) to make a determination. Other advantages include the ability of some fluorescent labels to fluoresce at different wavelengths with different intensities under different conditions. The latter is useful in determining whether or not the labelled ligand has penetrated into a cell, since the conditions, for example, pH, intracellularly versus extracellularly are quite different. For example, one can also look at lateral mobility, the passage of molecules into and out of cells. This is not possible with radioactive labels. The intensity of some fluorescent labels also declines over time after binding, allowing one to measure binding kinetics with one label. One can also use a quenching ligand to reduce intensity, for example, where more than one fluorescent label has been used, to create a three dimensional structural/activity comparison of a receptor conformation.

### Procedures for Binding Label to Antibody

Methods known to those skilled in the art are used to bind the label to the antibody. Examples are provided in detail below. In general, the label is bound to the antibody at a site where the label does not sterically hinder binding of the antibody to PDI or the non-labeled antibody to PDI, usually through an amine group, using a protection-deprotection process. The site of attachment of the fluorescent label to the antibody is particularly important as compared with attachment of a radiolabel to the antibody since a radiolabel usually consists of one or two atoms while the fluorescent probe may be of the same size as the antibody and therefore much more likely to interfere with binding to the PDI or the non-labeled first antibody. The label must also be bound so that the antibody does significantly interfere with or decrease the fluorescent intensity of the label.

In some cases, to avoid steric interference, the label is bound to the antibody through a spacer. In the preferred embodiment, the spacer consists of a two to seven methylene carbon chain which prevents the label from interfering with the binding activity of the ligand.

### Methods for Measuring Fluorescence

Analysis of intracellular events versus extracellular events is accomplished by choosing an appropriate fluorescent probe (e.g., fluorescein) which emits different fluorescent intensities depending on the pH of the environment, labeling the specific ligand with the label, and exposing the cells to the labelled ligand under conditions at which binding can occur. The pH of the inside of the cell is generally known (usually in the range of 7.4). The pH of the extracellular environment can be manipulated as desired to produce a difference in fluorescent intensity. For example, fluorescein has a low fluorescent intensity at pH 6.0 and optimum fluorescent intensity at pH 8.1. The fluorescein is ionized and the different intensities measured to quantitate the amount of intracellular versus extracellular binding.

In conventional confocal fluorescence microscopy, a specimen is illuminated point-by-point by an objective lens. (The point size measured at an Airy disk is less than one micron.) The emission light emerging from the illuminated point is collected by the same objective lens, passes through a selection diaphragm which is conjugate to the original illumination diaphragm and eliminates scattered light from all other regions of the specimen. The emission light finally is detected by a photomultiplier. By scanning the illuminated point in the object plane, a 2-D image can be sequentially determined. Additional shifting of the objective in the z-direction allows a 3-D image to be reconstructed. The fact that only the directly illuminated point contributes to image information is a guarantee of a resolution higher than that of conventional recording methods, and it enables 3-D resolution by topographical means.

All documents mentioned herein are incorporated herein by reference, in their entirety. The following example illustrates embodiments of the invention, but should not be viewed as limiting the scope of the invention.

### Exemplification

### Materials and Methods of the Examples

Subjects. Blood samples, fibroblast cells, and photos were obtained after informed consents according to protocols approved by the IRBs of the Johns Hopkins University and the King Khaled Eye Specialist Hospital of the Kingdom of Saudi Arabia.

RT-PCR, candidate gene screening, and Taqman assays. Primary fibroblast cell lines from a heterozygous carrier and a homozygous affected individual were established. Total RNA was extracted using trizol reagent (Invitrogen) and purified with the RNeasy micro kit (Qiagen) according to manufacturer's instructions. cDNA was prepared with Superscript reverse transcriptase (Invitrogen™). All known genes and putative transcripts in the region between markers D 14S 1014 and D 14S301 on chr. 14 were identified using the Human Genome Browser (http://genome.ucsc.edu/). The candidate genes in the area were prioritized according to their function and expression pattern with relevance to CLSD. Primers were designed to amplify and sequence the entire coding regions of these genes from cDNA. PCR was performed using Platinum Taq (Invitrogen™) and the PCR products were purified from solution using Shrimp Alkaline Phosphotase and Exonuclease I ((J.S. Biochemical), or from gel bands using QIAquick gel extraction kit (Qiagen). The Sequenase v. 2.0 DNA sequencing kit (U.S. Biochemical) and the ABI Prism 3700 fluorescent DNA analyzer (PE Biosystems) were used for direct sequencing of PCR products. A custom Taqman assay for the c.144T>C mutation was designed (Applied Biosystems) and 300 individuals were tested.

Protein sequence analysis and homology modeling. SEC23A protein sequences from ten species were obtained from NCBI and aligned using CLUSTAL W to determine the degree of conservation at the mutated amino acid position. A model of the F382L mutant human SEC23A protein was generated by structural homology modeling. Utilizing as the template a model of the wild type human SEC23A protein (UniProt AC Q 15436; SWISS-MODEL Repository http://swissmodel.expasy.org/repository), based on the crystal structure of Sec23 protein from S. cerevisiae (PDB 1M20) we modeled the effect of the mutation using SWISS-MODEL First Approach mode and viewed it using RasMol Version 2.7.2.1 (RasWin Molecular Graphics).

Cell culture and transfection. Primary fibroblast cell lines were derived from skin biopsy of an affected and carrier individuals. Control fibroblasts were obtained from ATCC (ATCC 2091). The fibroblasts were maintained in MEM media with 20% FBS.

Direct and indirect immunofluorescence microscopy. Fibroblasts (2.5×10⁵) were plated on glass cover slips in 6-well plates 24 hours prior to staining. Cells were washed briefly with PBS and fixed in 3% paraformaldehyde for 10 minutes at room temperature, then permeabilized in 0.5% Triton-X 100 for 3 minutes at room temperature. Primary antibodies for indirect IF were polyclonal rabbit antibodies to SARI (a gift form C. M. Mansbach, University of Tennessee, Memphis), SEC23 (a gift from F. Gorelick, Yale University), SEC24A, B, and C (a gift from J-P. Paccaud, Athelas S A) and rSec22b (a gift from Jesse Hay, University of Montana), SEC31 (a gift from A. Hubbard, Johns Hopkins University), SEC 13 (a gift from W. Hong, University of Singapore), LF-67 anti-COLA1A rabit serum (a gift from L. W. Fisher, NIH), and polyclonal mouse antibody to GM130 (a gift from C. Machamer, Johns Hopkins University). Secondary antibodies were Alexa Fluor 488-conjugated goat antibody to rabbit IgG conjugated to fluorescein isothioicyanate or Texas red goat antibody to mouse IgG. After staining the cells using the appropriate primary and secondary antibodies images were visualized and captured with a Nikon Eclipse 400 microscope.

Electron microscopy. Confluent fibroblast cell cultures were fixed in 2% glutaraldehyde in 0.1M phosphate buffer, gently scraped, centrifuged, post-fixed in osmium tetroxide, dehydrated, and embedded in Epon. Thin sections were sequentially stained with uranyl acetate and lead citrate and examined using a Philips electron microscope.

COL1A1 secretion assay. Primary fibroblasts were grown in 6-well plates until nearly confluent. Growth media was replaced with serum-free DMEM containing 10 mM HEPES and 50 ug/mL ascorbate-2-phosphate. 50 ug/mL cycloheximide was added as a control to some wells. Conditioned media was collected after 5 hours. Cells were washed twice with D-PBS and resuspended in 50 mM Tris pH 7.4, 150 mM NaCl, 10 mM EDTA, 1% Triton X-100, 0.1% SDS. Samples were run on 7% polyacrylamide gels under denaturing and reducing conditions after normalizing for total protein in each sample. Proteins were transferred to PVDF membranes and probed with LF-41 anti-COL1A1 rabbit serum²⁵.

In-vitro assays. A baculovirus expression vector of human SEC23A was created by insertion of a PCR product from a cDNA template (IMAGE #4821858) into the pFastBac1 plasmid (Invitrogen). The F382L mutation was subsequently introduced via primer-directed mutagenesis. The F382L SEC23A/hisSEC24D complex, wild-type SEC23A/hisSEC24D complex, and human SEC 13/hisSEC31A complex were purified from baculovirus-infected SF9 cells as describe²¹. Human SARIB cDNA (SARA2 gene) was subcloned into the pGEX-2T (GE Healthcare) vector and purified as described for hamster Sarla²¹. Liposome binding. assays using major-minor mix liposomes were performed essentially as described in²⁶ incubating at 37 C for 20 minutes prior to centrifugation. NIH-3T3 cells were permeabilized with digitonin²⁷ and used as donor ER membranes for in vitro vesicle formation reactions carried out as describe²¹.

Zebrafish expression analysis. Total RNA was extracted from Singapore wild-type zebrafish embryos of different stages and analyzed for sec23a expression using the SuperScript™ III One-Step RT-PCR System (Invitrogen™). A 1262 by cDNA fragment was amplified from 1 μg total RNA template over 35 step-cycles using primers 5′GAAGTGCTGCACCAACTATACAG and 5′AATGTGACCCTGCTGGCTGTG. A 561 by actin cDNA internal positive control fragment was co-amplified using primers 5′CGCACTCCTTCTTCACAACG and 5′AGGATCTTCATCACCTACTC. Digoxigenin-labeled antisense and sense RNA probes corresponding to the 3′ region of Sec23a cDNA (c.2341-c.2893 of GenBank #BC097063.1) were generated by cloning the 553 by cDNA fragment in pCRII®-TOPO® vector (Invitrogen) after amplification with primers 5′GCGCA00ATTCTGACAG and 5′CATTGTCATTTACCTCACCC, linearization with EcoRV and in vitro transcription using SP6 RNA polymerase, or linearization with BamHI and in vitro transcription using B T7 RNA polymerase, respectively. Whole-mount in situ hybridization was carried out as described previously”.

Zebrafish morpholino knockdown analysis. Two different translation-blocking antisense morpholinos specific to sec23a mRNA were designed (P:5′TGTCTCCTCAGAGACTCCCAGTCAG; and Q:5′CGCACCCTCTGGAATCTGAATCCTT). Singapore wildtype zebrafish embryos between 1-4 cell stage were injected with 8 ng of either morpholino P or Q. Live and Alcian blue stained 4 and 5 dpf hatchlings were observed under a brightfield microscope. All images were digitally taken using either a DP50 digital camera (Olympus) and processed using Adobe Photoshop version 7 software, or a MicroPublisher™ color digital camera (QImaging™) and processed using Image-Pro® Plus version 5 software (Media Cybemetics®).

### Examplification

### Example 1

### Demonstration of ER Distention by Accumulation of Secretory Proteins

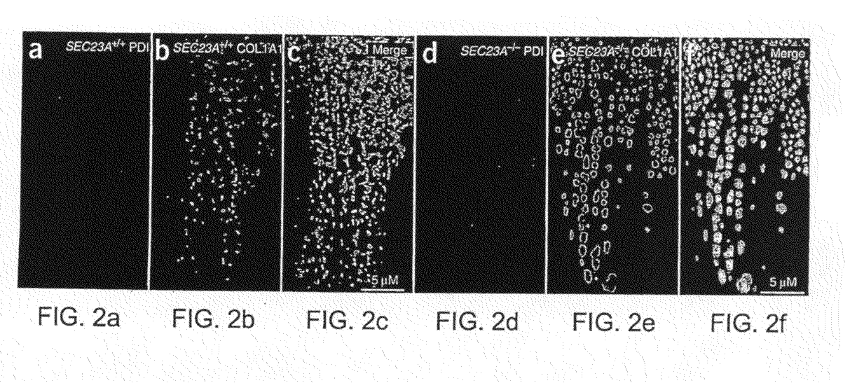

Based on the known biologic function of SEC23A, we predicted excessive accumulation of secretory proteins within the rough ER of the mutant fibroblasts. Using an antibody against the intralumenal ER chaperone PDI¹⁸ we were able to demonstrate abundant vacuolar structures in the homozygous mutant cells that were interpreted as distended ER (FIG. 2a, b). IF with procollagen antibody documented substantial accumulation within ER cistemae similar in morphology to those marked by PDI (FIG. 2c, d). IF with antibodies against SEC31 demonstrated diffuse cytoplasmic mislocahzation of this protein m the mutant fibroblasts, suggestive of abnormal COPII complex formation (FIG. 2e, f). Additional IF experiments with antibodies against SARI, SEC24A, SEC24B, SEC24C, SEC 13, SEC22B and GM130, a cis-Golgi matrix protein 19 did not demonstrate abnormal cellular phenotypes.

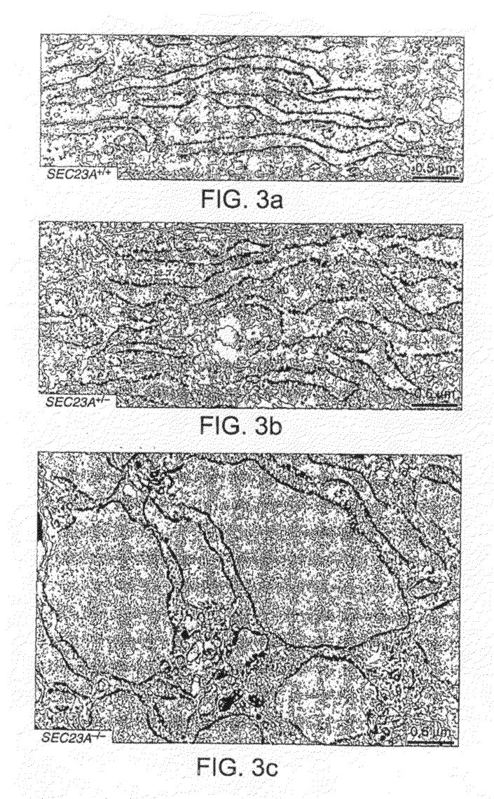

Wild-type and SEC23A F382L heterozygous and homozygous mutant fibroblasts were examined by thin section electron microscopy. In the vast majority of wild type cells the rough ER had a typical organization with narrow cisternae (FIG. 3a) with approximately 10% of cells showing mild focal dilatation of the ER. Approximately 35% of heterozygous fibroblast sections showed a moderate generalized dilatation of ER, suggesting a dominant-negative effect of the mutant SEC23A (FIG. 3b). More than 80% of the homozygous mutant cells showed cisternae of the ER greatly distended by accumulation of secretory material (FIG. 3c).

### Example 2

### Competent Collagen Secretion

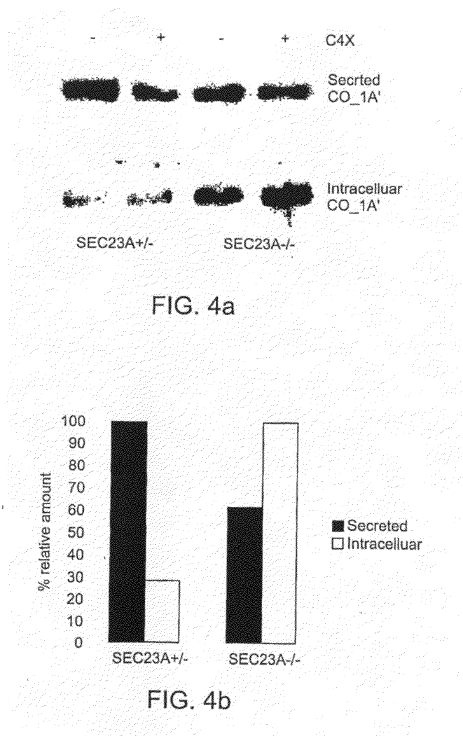

The skeletal phenotype of CLSD involves predominantly the membranous bones of the calvaria but, vertebral and pelvic bone defects and generalized joint hypermobility are also present. These connective tissue features suggested a potential secretory defect of COL1A1 and/or other related extracellular matrix proteins. A defect in collagen secretion suggested by IF (FIG. 2d) was further explored by immunoblot analysis of secreted and intracellular proteins (FIG. 4a, b). Homozygous mutant cells accumulated a significant pool of collagen that was not depleted during a 6.5 h incubation in the presence of cyclohexamide. These data documented that the SEC23A mutations results in a defect of the COPII secretory pathway. The fact that secretion of COL 1A1 is not completely blocked in the mutant fibroblasts suggests either the presence of a COPII-independent collagen export pathway or, more likely, compensation for SEC23A function by the highly homologous SEC23B protein²⁰.

### Example 3

### Functional Consequence of the F382L Mutation

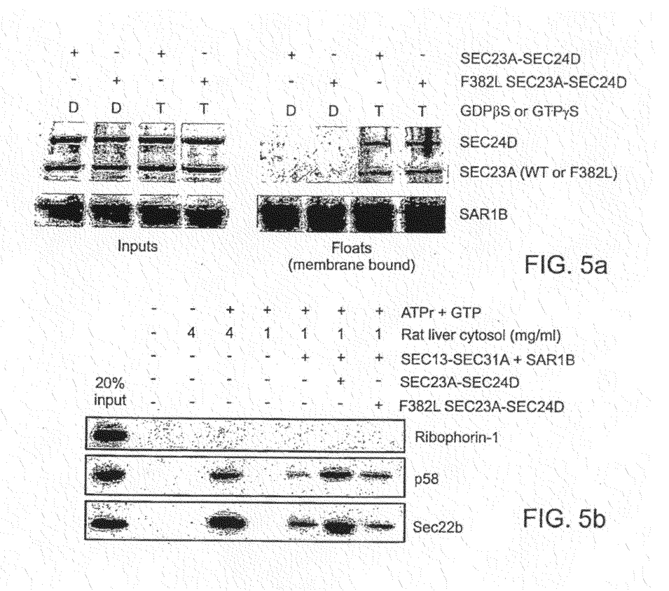

In order to assess the functional consequence of the F382L mutation, we purified recombinant F382L SEC23A in complex with histidine-tagged SEC24D from baculovirus-infected insect cells. We found that the F382L SEC23A/SEC24D complex bound to synthetic membranes in the presence of activated SARI at a level similar to the wild-type protein (FIG. 5a). We next tested the function of F382L SEC23A in an in vitro vesicle formation assay²¹. Under conditions requiring the addition of purified SEC23/SEC24 complex for the formation of cargo-containing vesicles from donor ER membranes, F382L SEC23A/SEC24D was not capable of providing wild-type function (FIG. 5b). Our data thus demonstrate that F382L SEC23A is able to bind SAM and SEC24D, but has lost an important aspect of its function in vesicle budding, likely related to formation of a competent complex with SEC 13/SEC31 (FIG. 2.e, f).

### Example 4

### Phenotypic Consequences of sec23a Loss of Function in Zebrafish

Next, we determined the developmental expression pattern of the zebrafish (Danio rerio) ortholog using reverse-transcription PCR (RT-PCR) and whole mount in situ hybridization analyses, and performed a morpholino-mediated knockdown analysis to assess the phenotypic consequences of sec23a loss of function.

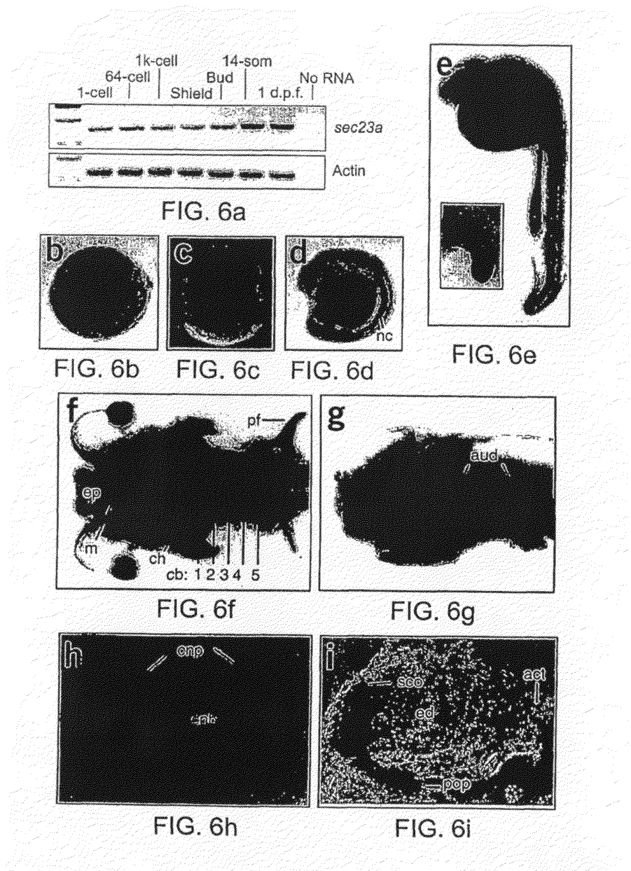

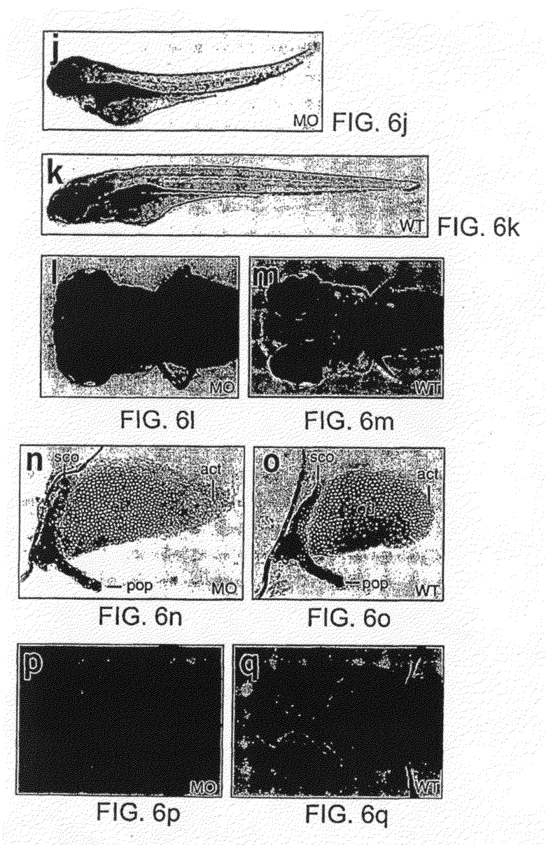

RT-PCR analysis of 16 embryonic stages detected sec23a transcript from as early as the 1-cell stage, indicating the presence of maternal transcript (FIG. 6a). However, transcript levels were insufficient to be detected by whole mount in situ hybridization analysis (FIG. 6b, c) until the 12-somite stage (FIG. 6d) when expression in the developing potochord was observed. Notochord expression was strongest in the 1 day post-fertilization (dpf) embryo (FIG. 6e) and was undetectable by 2 dpf (not shown). At this stage, however, some expression in the developing head cartilages could be observed (not shown). In 3 dpf embryos, strong expression in all major neurocrania( and viscerocranial cartilages of the head was observed (FIG. 6f, g). Additionally, weak expression could be detected in the cranial project and-bulge of the otic vesicle (FIG. 6h). Furthermore, distinct sec23a expression was observed in the scapulocoracoid and postcoracoid processes of the pectoral fin, as well as the distal edge of the endoskeletal disc, but not in the surrounding actinotrichs (FIG. 6i).

Loss-of-function analysis using 2 morpholinos targeting different regions immediately upstream of the translation start site, and complementary to sec23a but not sec23b, yielded identical phenotypes. Morphant 5 dpf hatchlings showed a reduced body length and a dorsally oriented curvature compared to uninjected hatchlings (FIG. 6j, k), and exhibited reduced and upward swimming motion, suggesting the possibility of an effect on vertebral development analogous to the scoliosis in CLSD. Unfortunately, the lack of ossified skeleton in 5-7 dpf hatchlings precluded a more detailed bone analysis, and morphants generally did not survive beyond 9 dpf to enable analysis at later stages. Kinking of the pectoral fins was also observed in morpholino-injected hatchlings from 4 dpf onwards (FIG. 6l, m). Alcian blue staining of pectoral fins of 5 dpf morphants revealed an elongated actinotrich region compared to wildtype, where the entire endoskeletal disc appeared intact (FIG. 6n, o). Given the absence of sec23a expression in the actinotrichs, the results suggest that actinotrich outgrowth is regulated by sec23a expression at the distal edge of the endoskeletal disc.

As expected, sec23a morphants exhibited morpholino dosage-dependent malformations of all major neurocranial and viscerocranial cartilage structures, including the ethmoid plate, parachordal, and auditory capsule, the Meckel's cartilage, ceratohyal, and ceratobranchial arches (FIG. 6p, q). Although detailed characterization of these cranial defects is difficult in the absence of an adult morphant or gemiline mutant, the observed changes and weak Alcian blue staining suggest a generalized cartilage hypoplasia, most likely due to aberrant or delayed chondrogenesis and/or delayed deposition of extracellular matrix molecules. These observations are consistent with a role for sec23a in craniofacial development, and reflect the observed craniofacial defects in CLSD.

### REFERENCES

1. Boyadjiev, S. A. et al. A novel dysmorphic syndrome with open calvarial sutures and sutural cataracts maps to chromosome 14g13-q21. Hum Genet 113, 1-9 (2003).
2. Duden, R. ER-to-Golgi transport: COP I and COP U function (Review). Mol Membr Biol 20, 197-207 (2003).
3. Schekman, R. & Orci, L. Coat proteins and vesicle budding. Science 271, 1526-33 (1996).
4. Novick, P., Field, C. & Schekman, R. Identification of 23 complementation groups required for post-translational events in the yeast secretory pathway. Cell 21, 205-15 (1980).
5. Novick, P. & Schekman, R. Secretion and cell-surface growth are blocked in a temperature-sensitive mutant of Saccharomyces cerevisiae. Proc Natl Acad Sci USA 76, 1858-62 (1979).
6. Novick, P. & Schekman, R. Export of major cell-surface proteins is blocked in yeast secretory mutants. J Cell Biol 96, 541-7 (1983).
7. Barlowe, C. et al. COPII: a membrane coat formed by Sec proteins that drive vesicle budding from the endoplasmic reticulum. Cell 77, 895-907 (1994).
8. Antonny, B. & Schekman, R. ER export: public transportation by the COPA coach. Curr Opin Cell Biol 13, 43 8-43 (2001).
9. Miller, E., Antonny, B., Hamamoto, S. & Schekman, R. Cargo selection into COPII vesicles is driven by the Sec24p subunit Embo J 21, 6105-13 (2002).
10. Miller, E. A. et al. Multiple cargo binding sites on the COPII subunit Sec24p ensure capture of diverse membrane proteins into transport vesicles. Cell 114, 497-509 (2003).
11. Nakano, A., Brada, D. & Schekman, R. A membrane glycoprotein, Sec 12p, required for protein transport from the endoplasmic reticulum to the Golgi apparatus in yeast. J Cell Biol 107, 851-63 (1988).
12. Barlowe, C. & Schekman, R SEC 12 encodes a guanine-nucleotide-exchange factor essential for transport vesicle budding from the ER. Nature 365, 347-9 (1993).
13. Aridor, M., Weissman, J., Bannykh, S., Nuoffer, C. & Balch, W. E. Cargo selection by the COPE budding machinery during export from the ER J Cell Biol 141, 61-70 (1998).
14. Pagano, A. et al. Sec24 proteins and sorting at the endoplasmic reticulum. J Biol Chem 274, 7833-40 (1999).
15. Antony, B., Madden, D., Hamamoto, S., Orci, L. & Schekman, R. Dynamics of the COPA coat with GTP and stable analogues. Nat Cell Biol 3, 531-7 (2001).
16. Bi, X., Corpina, R. A. & Goldberg, J. Structure of the Sec23/24-Sarl pre-budding complex of the COPII vesicle coat. Nature 419, 271-7 (2002).
17. Roberts, B., Clucas, C. & Johnstone, I. L. Loss of SEC-23 in Caenorhabditis elegans causes defects in oogenesis, morphogenesis, and extracellular matrix secretion. Mol Biol Cell 14, 4414-26 (2003).
18. Bottomley, M. J., Batten, M. R., Lumb, R. A. & Bulleid, N. J. Quality control in the endoplasmic reticulum: PDI mediates the ER retention of unassembled procollagen C-propeptides. Curr B161 11, 1114-8 (2001).
19. Nakamura, N. et al. Characterization of a cis-Golgi matrix protein, GM130. J Cell Biol 131, 1715-26 (1995).
20. Paccaud, J. P. et al. Cloning and functional characterization of mammalian homologues of the COPII component Sec23. Mol Biol Cell 7,1535-46 (1996).
21. Kim, J., Hamamoto, S., Ravazzola, M., Orci, L. & Schekman, R. Uncoupled packaging of amyloid precursor protein and presenilin 1 into coat protein complex II vesicles. J Biol Chem 280, 7758-68 (2005).
22. Jones, B. et al. Mutations in a Sarl GTPase of COPH vesicles are associated with lipid absorption disorders. Nat Genet 34, 29-31 (2003).
23. Tiller, G. E. et al. A recurrent RNA-splicing mutation in the SEDL gene causes Xlinked spondyloepiphyseal dysplasia tarda. Am J Hum Genet 68, 1398-407 (2001).
24. Zhang, B. et al. Bleeding due to disruption of a cargo-specific ER-to-Golgi trasport complex. Nat Genet 34, 220-5 (2003).
25. Fisher, L. W., Lindner, W., Young, M. F. & Termine, J. D. Synthetic peptide antisera: their production and use in the cloning of matrix proteins. Connect Tissue Res 21, 43-8; discussion 49-50 (1989).
26. Matsuoka, K. & Schekman, R. The use of liposomes to study COPII- and COPI-coated vesicle formation and membrane protein sorting. Methods 20, 417-28 (2000).
27. Wilson, R. et al. The translocation, folding, assembly and redox-dependent degradation of secretory and membrane proteins in semi-permeabilized mammalian cells. Biochem J 307 (Pt 3), 679-87 (1995).
28. Ben, J., Jabs, E. W. & Chong, S. S. Genomic, cDNA and embryonic expression analysis of zebrafish IRF6, the gene mutated in the human oral clefting disorders Van der Woude and popliteal pterygium syndromes. Gene Expr Patterns 5, 629-38 (2005).
29. Bulleid, N. J. Protein Disulfide Isomerase: Role in Biosynthesis of Secretory Proteins (1993) Advances in Protein Chemistry 44, 125-50
30. Noiva, R. Enzymatic Catalysis of Disulfide Formation (1994) Protein Expression and Purification 5, 1-13
31. Freedman, R. B., Hirst, T. R. and Tuite, M. F. Protein disulfide isomerase: building bridges in protein folding (1994) Trends in Biochemical Sciences 19, 331-336

The foregoing description of the invention is merely illustrative thereof, and it is understood that variations and modifications can be effected without departing from the scope of spirit of the invention as set forth in the following claims.

I claim:

1. A method of diagnosing a patient with a disease or disorder that is associated with defective Endoplasmic Reticulum-to-Golgi trafficking of proteins comprising:
a) culturing primary fibroblast cells from a patient;
b) plating the cultured fibroblast cells on a transparent structural support and treating the cells with a permeabilizing agent;
c) incubating the cells with a first antibody of animal species A which is directed to protein disulfide isomerase;
d) incubating the cells with a second antibody of animal species B which is conjugated with a fluorescent label and directed to the first antibody of animal species A; and
e) visualizing the cells under a fluorescence microscope and identifying distended Endoplasmic Reticulum with abundant vacuolar structures wherein the presence of distended Endoplasmic Reticulum is diagnostic of a disease or disorder associated with defective Endoplasmic Reticulum-to-Golgi trafficking of proteins for the patient.

2. The method of claim 1 wherein the RNA of the fibroblast cells is extracted from the cells and quantitative RT-PCR analysis is conducted for a specific isoform of the X-Box-Binding Protein 1 (XBP1) gene to determine Unfolded Protein response (UPR) activation, which if UPR is not activated, an Endoplasmic Reticulum-to Golgi disease or disorder is confirmed.

3. The method of claim 1 wherein the disease or disorder is Cranio-lenticulo-sutural dysplasia.

4. The method of claim 1 wherein the transparent structural support is a glass cover slip.

5. The method of claim 1 wherein the first antibody of animal species A is a rabbit antibody.

6. The method of claim 1 wherein the second antibody of animal species B is a goat antibody.

7. The method of claim 1 wherein the fluorescent label is fluorescein isothiocyanate.

8. The method of claim 1 wherein the fluorescent label is Texas red goat antibody.

9. The method of claim 1 wherein first antibody of animal species A which is directed to protein disulfide isomerase is commercially available.

10. The method of claim 1 wherein the first antibody of animal species A which is directed to protein disulfide isomerase is polyclonal.

11. The method of claim 1 wherein the first antibody of animal species A which is directed to protein disulfide isomerase is monoclonal.

12. The method of claim 1 wherein second antibody of animal species B which is conjugated with a fluorescent label is polyclonal.

13. The method of claim 1 wherein second antibody of animal species B which is conjugated with a fluorescent label is monoclonal.

14. A kit for diagnosing a patient with a disease or disorder that is associated with defective Endoplasmic Reticulum-to-Golgi trafficking of proteins comprising a first antibody of animal species A which is directed to protein disulfide isomerase, a second antibody of animal species B which is conjugated with a fluorescent label and directed to the first antibody of animal species A, a permeabilizing agent and instructions for use in performing the diagnostic test.

15. A method of diagnosing a patient with a disease or disorder that is associated with defective Endoplasmic Reticulum-to-Golgi trafficking of proteins comprising:
a. culturing primary fibroblast cells from a patient;
b. plating the cultured fibroblast cells on a transparent structural support and treating the cells with a permeabilizing agent;
c. incubating the cells with a fluorescent labeled antibody which is directed to protein disulfide isomerase; and
d. visualizing the cells under a fluorescence microscope and identifying distended Endoplasmic Reticulum with abundant vacuolar structures wherein the presence of distended Endoplasmic Reticulum is diagnostic of a disease or disorder associated with defective Endoplasmic Reticulum-to-Golgi trafficking of proteins for the patient.

16. The method of claim 15 wherein the RNA of the fibroblast cells is extracted from the cells and quantitative RT-PCR analysis is conducted for a specific isoform of the X-Box-Binding Protein 1 (XBP1) gene to determine Unfolded Protein response (UPR) activation, which if UPR is not activated, a Endoplasmic Reticulum-to Golgi disease or disorder is confirmed.
